(19) United States
(12) Patent Application Publication
(10) Pub. No.: US 2009/0257956 A1
(43) Pub. Date: Oct. 15, 2009

(54) Treatment Method for Treatment of Erectile Dysfunction and Premature Ejaculation

(76) Inventors: Ramsey Sallis, St. Peters (AU); Quoc Huan Ha, Costa Mesa, CA (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 12/422,810

(22) Filed: Apr. 13, 2009

Related U.S. Application Data
(63) Continuation-in-part of application No. 12/178,913, filed on Jul. 24, 2008.
Application No. 12/178,913 is a continuation-in-part of application No. 10/351,049, filed on Jan. 24, 2003, now Pat. No. 7,405,222.
(60) Provisional application No. 60/351,634, filed on Jan. 25, 2002.

(51) Int. Cl. A61K 49/00; A61P 15/00

(52) U.S. Cl. 424/9.2

(57) ABSTRACT

A method for a same visit treatment for erectile dysfunction and premature ejaculation. The method includes: performing a diagnostic on the patient using physical conditions of the patient; determining a diagnostic group of the patient, wherein each diagnostic group has corresponding conditions to the physical conditions of the patient; identifying an age group of the patient; selecting a first test dose of a pharmaceutical composition, which corresponds to the age group and the diagnostic group of the patient, wherein the pharmaceutical composition is a solution with PGE1, papaverine, phentolamine, and atropine; administrating the first test dose to the patient; observing the physical conditions of the patient after the administration of the first test dose; determining a result group of the patient after observing the physical conditions of the patient; and prescribing a same visit dosage for the patient based on the result group.

### CROSS-REFERENCE TO RELATED APPLICATIONS

This application is a continuation-in-part and claims priority to U.S. patent application entitled, “METHODS AND COMPOSITIONS FOR TREATMENT OF ERECTILE DYSFUNCTION,” filed Jul. 24, 2008, having Ser. No. 12/178,913, which is a continuation-in-part and claims priority to U.S. patent application entitled, “METHODS AND COMPOSITIONS FOR TREATMENT OF ERECTILE DYSFUNCTION,” filed Jan. 24, 2003, having Ser. No. 10/351,049, now issued as U.S. Pat. No. 7,405,222, which claims priority to U.S. provisional patent application entitled, “METHODS AND COMPOSITIONS FOR TREATING MALE ERECTILE DYSFUNCTION,” filed Jan. 25, 2002, having Ser. No. 60/351,634, the disclosures of which are hereby incorporated by reference in their entirety.

### FIELD OF THE INVENTION

This invention relates generally to the fields of medicine and urology. More particularly, the invention relates to methods and compositions for treatment of male erectile dysfunction.

### BACKGROUND OF THE INVENTION

Sexual dysfunction in both males and females has received a significant amount of popular attention with the greater concerns directed to the particular male problems of erectile capacity and penetration ability. Male sexual dysfunction associated with impotence is generally defined as the inability to attain or sustain an erection satisfactory for normal coitus. Prevalence of erectile problems, including premature ejaculation, has been estimated to be surprisingly high, perhaps as much as 45% worldwide. This is a particular concern among the over-50 male population in the United States, where estimates of impotency range from 18 to 75% for this age group.

Medical intervention for treatment of erectile dysfunction is becoming increasingly common. Surgical procedures, penile implants and pharmacological agents have been developed for treatment of male erectile dysfunction in recent years.

Surgical interventions employing penile implants and various patient-controlled mechanical devices are not uniformly successful nor are they appropriate in most situations. A serious disadvantage of treating erectile dysfunction with a surgical implant is the necessity to irreversibly damage the erectile tissues of the penis.

Pharmacological methods have been developed to treat male erectile dysfunction. The mechanism or erection is dependent on adequate blood flow to the spongy cavernous tissue of the penis, in paired spaces known as the corpora cavernosa. Control of blood flow to the corpora cavernosa involves the process of vasodilation of the cavernosal arteries.

It is now recognized that vasodilators acting on the penile arteries can be beneficial to achieving an erection. Orally administered vasodilators are currently the most widely accepted treatments for male erectile dysfunction. Such vasodilators include sildenafil. This compound is taken orally about one hour prior to intercourse because instant erection is not achieved with this drug. Unfortunately, this delay may lead to inconvenience for patients seeking a more rapid response. While effective, this product may cause undesirable side effects such as headache, flushing, dyspepsia and in some cases abnormal vision such as increased sensitivity to light. Of greater concern, this drug may exacerbate pre-existing heart conditions. Furthermore, sildenafil must be taken on an empty stomach and must not be taken with alcohol.

A more rapid response in producing an erection has been found for sildenafil applied sublingually or intranasally. The response is often within five minutes, and the drug is asserted to have fewer side effects than when administered orally. While reducing side effects, the drug nevertheless enters the circulatory system and is able to exert vasodilatory effects in areas other than the penis. Following the nasal or sublingual route of administration, undesirable side effects can still be produced, some of which may be of medical concern.

Recognition of the problems associated with systemic administration of vasodilators has led to development of methods for local delivery of vasodilators to the tissues of the penis. For example, a semi-solid vehicle containing a vasodilator for insertion into the urethra has been formulated and shown to be effective in stimulating erection. Disadvantages of this method are the requirement for administration into the urethra, contributing to transient burning or tingling effects reported in some cases, and raising the possibility of urethral infection and scarring. This product is ineffective in up to 60% of patients.

An alternative and more direct route for delivery of vasodilators is injection into the corpus cavernosum of the penis. This “intracavernosal” route has been successfully used to deliver one or more vasodilators to achieve erections, and has led to the development of a treatment regimen known as intracavernosal pharmacotherapy (ICP).

Studies have led to the discovery of several vasodilators capable of provoking an erection. The use of papaverine for male erectile dysfunction has been reported. Papaverine is a vascular intracellular smooth muscle relaxant acting by its inhibitory effect on cyclic mononucleotide phosphodiesterases. However papaverine as monotherapy had an unpredictable response, and the drug must be administered in large volumes. Additionally, significant side effects associated with papaverine include the potential for prolonged erection and fibrosis of the corpus cavernosum.

Another vasodilator that has been used to achieve erection is phentolamine. Phentolamine alone does not reliably produce an erection sufficient for intercourse.

Prostaglandin E1 (PGE1) is yet another vasodilator that effects an erection through its action as an alpha-adrenergic blocker. PGE1 has been formulated into suppositories for insertion into the urethra of men suffering from erectile dysfunction. As described above, this method of administration is disadvantageous due to risk of urethral infection and fibrosis. PGE1 has a short half life compared with papaverine and phentolamine. Intracavernosal injection of PGE1 can cause an erection of dose-dependent duration but, in a trial of this method, was associated with significant pain in a large percentage (up to 40%) of the subjects.

Several combinations of vasodilators have been used for treatment of impotence by the intracavernosal route. Success has been reported in a clinical study using a combination of papaverine and phentolamine in patients having vascular causes of impotence mostly associated with diabetes or atherosclerosis. Clinical success has also been reported in men with erectile dysfunction of neurogenic origin using a combination of papaverine, phentolamine and PGE1. The three-drug mixture has been regarded as more effective than PGE1 alone in inducing an erectile response, with a decreased incidence of pain. This drug combination also decreases the latency between injection and erection and allows a reduction in the total papaverine dose, reducing the risk of prolonged erection and potential incidence of fibrosis.

The collective experience of urologists reviewing clinical trials and treating patients with erectile dysfunction has led to an appreciation that despite impressive successes with some patients, no one therapy or composition can be applied across-the-board to treat all erectile problems.

### SUMMARY OF THE INVENTION

The foregoing needs are met, to a great extent, by the present invention, wherein in one aspect a method is provided for a same visit treatment of achieving and maintaining male erectile response regardless of state of mind, occurrence of ejaculation, or lack of sexual arousal of a patient. In another aspect a method is provided for same visit and follow-up treatments for erectile dysfunction. In another aspect a method is provided by same visit and follow-up treatments for premature ejaculation. In yet another aspect a method is provided for the treatment of resistant erectile dysfunction.

In accordance with one embodiment of the present invention, a method for a same visit treatment of achieving and maintaining male erectile response regardless of state of mind, occurrence of ejaculation, or lack of sexual arousal of a patient is provided. The method includes: (a) performing a diagnostic on the patient using physical conditions of the patient, including blood flow rate, rigidity of erection, a sustainability of an erection, occurrence of morning erection, duration of erectile dysfunction, and an ability to penetrate; (b) determining a diagnostic group of the patient, including premature ejaculation group, mild erectile dysfunction, moderate erectile dysfunction, and severe erectile dysfunction, wherein each diagnostic group has corresponding conditions to the physical conditions of the patient; (c) identifying an age group of the patient; (d) selecting a first test dose of a pharmaceutical composition, which corresponds to the age group and the diagnostic group of the patient, wherein the pharmaceutical composition is a solution with PGE1, papaverine, phentolamine, and atropine; (e) administrating the first test dose of the pharmaceutical composition to the patient; (f) observing the physical conditions of the patient after the administration of the first test dose, including the rigidness of the penis of the patient and whether the patient is able to penetrate; (g) determining a result group of the patient after observing the physical conditions of the patient; and (h) prescribing a same visit dosage for the patient based on the result group.

In accordance with one another embodiment of the invention, a method for a same visit treatment and a follow-up treatment of erectile dysfunction regardless of state of mind, occurrence of ejaculation, or lack of sexual arousal of a patient is provided. The method includes: (a) performing a diagnostic on the patient using physical conditions of the patient, including blood flow rate, rigidity of erection, a sustainability of an erection, occurrence of morning erection, duration of erectile dysfunction, and an ability to penetrate; (b) determining a diagnostic group of the patient, including premature ejaculation group, mild erectile dysfunction, moderate erectile dysfunction, and severe erectile dysfunction, wherein each diagnostic group has corresponding conditions to the physical conditions of the patient; (c) identifying an age group of the patient; (d) selecting a first test dose of a pharmaceutical composition, which corresponds to the age group and the diagnostic group of the patient, wherein the pharmaceutical composition is a solution with PGE1, papaverine, phentolamine, and atropine; (e) administrating the first test dose of the pharmaceutical composition to the patient; (f) observing the physical conditions of the patient after the administration of the first test dose, including the rigidness of the penis of the patient and whether the patient is able to penetrate; (g) determining a result group of the patient after observing the physical conditions of the patient; (h) prescribing a same visit dosage for the patient based on the result group; (i) observing the physical conditions of the patient after the administration of the same visit dosage, including the rigidness of the penis of the patient and whether the patient is able to penetrate; (j) determining a follow-up visit result group of the patient after observing the physical conditions of the patient; and (k) prescribing a follow-up adjustment dosage for the patient based on the follow-up result group.

In accordance with yet another embodiment of the invention, a method for a same visit treatment and a follow-up treatment for resistant erectile dysfunction regardless of state of mind, occurrence of ejaculation, or lack of sexual arousal of a patient is provided. The method includes the steps of: (a) performing a diagnostic on the patient using physical conditions of the patient, including blood flow rate, rigidity of erection, a sustainability of an erection, occurrence of morning erection, duration of erectile dysfunction, and an ability to penetrate; (b) determining a diagnostic group of the patient, including premature ejaculation group, mild erectile dysfunction, moderate erectile dysfunction, and severe erectile dysfunction, wherein each diagnostic group has corresponding conditions to the physical conditions of the patient; (c) identifying an age group of the patient; (d) selecting a first test dose of a pharmaceutical composition, which corresponds to the age group and the diagnostic group of the patient, wherein the pharmaceutical composition is a solution with PGE1, papaverine, phentolamine, and atropine; (e) administrating the first test dose of the pharmaceutical composition to the patient; (f) observing the physical conditions of the patient after the administration of the first test dose, including the rigidness of the penis of the patient and whether the patient is able to penetrate; (g) determining a result group of the patient after observing the physical conditions of the patient; (h) prescribing a same visit dosage for the patient based on the result group; (i) observing the physical conditions of the patient after the administration of the same visit dosage, including the rigidness of the penis of the patient and whether the patient is able to penetrate; (j) determining a follow-up visit result group of the patient after observing the physical conditions of the patient; (k) prescribing a follow-up adjustment dosage for the patient based on the follow-up result group; (l) observing the physical conditions of the patient after the administration of 100 units of a solution of PGE1, papaverine, phentolamine, and atropine in the amount of 6 μg/ml, 10.5 mg/ml, 2.0 mg/ml and 0.15 mg/ml, respectively, including the rigidness of the penis of the patient and whether the patient is able to penetrate; (m) determining a resistant erectile dysfunction result group of the patient after observing the physical conditions of the patient; and (n) prescribing a first adjustment dosage for resistant erectile dysfunction for the patient based on the resistant erectile dysfunction result group.

In accordance with another embodiment of the present invention, a method for a same visit treatment and follow-up treatments for resistant erectile dysfunction regardless of state of mind, occurrence of ejaculation, or lack of sexual arousal of a patient is provided. The method includes the steps of: (a) performing a diagnostic on the patient using physical conditions of the patient, including blood flow rate, rigidity of erection, a sustainability of an erection, occurrence of morning erection, duration of erectile dysfunction, and an ability to penetrate; (b) determining a diagnostic group of the patient, including premature ejaculation group, mild erectile dysfunction, moderate erectile dysfunction, and severe erectile dysfunction, wherein each diagnostic group has corresponding conditions to the physical conditions of the patient; (c) identifying an age group of the patient; (d) selecting a first test dose of a pharmaceutical composition, which corresponds to the age group and the diagnostic group of the patient, wherein the pharmaceutical composition is a solution with PGE1, papaverine, phentolamine, and atropine; (e) administrating the first test dose of the pharmaceutical composition to the patient; (f) observing the physical conditions of the patient after the administration of the first test dose, including the rigidness of the penis of the patient and whether the patient is able to penetrate; (g) determining a result group of the patient after observing the physical conditions of the patient; (h) prescribing a same visit dosage for the patient based on the result group; (i) observing the physical conditions of the patient after the administration of the same visit dosage, including the rigidness of the penis of the patient and whether the patient is able to penetrate; (j) determining a follow-up visit result group of the patient after observing the physical conditions of the patient; (k) prescribing a follow-up adjustment dosage for the patient based on the follow-up result group; (l) observing the physical conditions of the patient after the administration of 100 units of a solution of PGE1, papaverine, phentolamine, and atropine in the amount of 6 μg/ml, 10.5 mg/ml, 2.0 mg/ml and 0.15 mg/ml, respectively, including the rigidness of the penis of the patient and whether the patient is able to penetrate; (m) determining a resistant erectile dysfunction result group of the patient after observing the physical conditions of the patient; (n) prescribing a first adjustment dosage for resistant erectile dysfunction for the patient based on the resistant erectile dysfunction result group; (o) observing the physical conditions of the patient after the administration of 100 units of a solution of PGE1, papaverine, phentolamine, and atropine in the amount of 10 μg/ml, 6.0 mg/ml, 2.5 mg/ml and 0.05 mg/ml, respectively, including the rigidness of the penis of the patient and whether the patient is able to penetrate; (p) determining a second resistant erectile dysfunction result group of the patient after observing the physical conditions of the patient; and (q) prescribing a second adjustment dosage for resistant erectile dysfunction for the patient based on the second resistant erectile dysfunction result group.

In accordance with yet another embodiment of the invention, a method for a same visit treatment and follow-up treatment for premature ejaculation regardless of state of mind, occurrence of ejaculation, or lack of sexual arousal of a patient is provided. The method includes the steps of: (a) performing a diagnostic on the patient using physical conditions of the patient, including blood flow rate, rigidity of erection, a sustainability of an erection, occurrence of morning erection, duration of erectile dysfunction, and an ability to penetrate; (b) determining a diagnostic group of the patient, including premature ejaculation group, mild erectile dysfunction, moderate erectile dysfunction, and severe erectile dysfunction, wherein each diagnostic group has corresponding values to the physical conditions of the patient; (c) identifying an age group of the patient; (d) selecting a first test dose of a pharmaceutical composition, which corresponds to the age group and the diagnostic group of the patient, wherein the pharmaceutical composition is a solution with PGE1, papaverine, phentolamine, and atropine; (e) administrating the first test dose of the pharmaceutical composition to the patient; (f) observing the physical conditions of the patient after the administration of the first test dose, including the rigidness of the penis of the patient and whether the patient is able to penetrate; (g) determining a result group of the patient after observing the physical conditions of the patient; (h) prescribing a same visit dosage for the patient based on the result group; (i) observing the physical conditions of the patient after the administration of the same visit dosage, including the rigidness of the penis of the patient and whether the patient is able to penetrate; (j) determining a follow-up visit result group of the patient after observing the physical conditions of the patient; and (k) prescribing a follow-up adjustment dosage for the patient based on the follow-up result group.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of constructions and to the arrangements of the components set forth in the following description or illustrated in the drawings. The invention is capable of embodiments in addition to those described and of being practiced and carried out in various ways. Also, it is to be understood that the phraseology and terminology employed herein, as well as in the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present inventions. It is important, therefore, that the claims be regarded as including such equivalent constructions insofar as they do not depart from the spirit and scope of the present invention.

### BRIEF DESCRIPTION OF THE DRAWINGS

The invention is pointed out with particularity in the appended claims. The above and further advantages of this invention may be better understood by referring to the following description, taken in conjunction with the accompanying drawings, in which:

FIG. 1 is a chart illustrating a portion of the inventive method involving determining a first test dose for patients with premature ejaculation or erectile dysfunction.

FIG. 2 is a chart illustrating a portion of the inventive method involving a same visit modification of the first test dose for erectile dysfunction.

FIG. 3 is a chart illustrating a portion of the inventive method involving a same visit modification of the first test dose prescription for premature ejaculation.

FIG. 4 is a chart illustrating a portion of the inventive method involving a follow-up prescription adjustment for erectile dysfunction.

FIG. 5. is a chart illustrating a portion of the inventive method involving a first dosage adjustment for resistant erectile dysfunction.

FIG. 6 is a chart illustrating a portion of the inventive method involving a second dosage adjustment for resistant erectile dysfunction.

FIG. 7 is a chart illustrating a portion of the inventive method involving a follow-up prescription adjustment for premature ejaculation.

FIG. 8 is a flow diagram of a portion of the inventive method illustrating the steps of using the charts in FIGS. 1-4 and 7.

FIG. 9 is a flow diagram of a portion of the inventive method illustrating the steps of using the charts in FIG. 5.

FIG. 10 is a flow diagram of a portion of the inventive method illustrating the steps of using the charts in FIG. 6.

FIG. 11 is a chart illustrating a portion of the inventive method involving a non-refrigerated formula.

### DETAILED DESCRIPTION

The invention provides a method of treating sexual dysfunction in a male mammal. A pharmaceutical composition of one or more agents is administered by ICP in an amount effective to cause the male to sustain an erection. The method includes an assessment of the general, physical and psychological condition of the male. A test dose of a pharmaceutical composition is formulated, guided by information obtained in the assessment step. A test dose of the formulated composition is administered to the subject by ICP. The erection characteristics of the male are observed following administration of the test dose. The composition may be subsequently adjusted to provide an effective erection.

The method is suitable for use in any male mammal capable of sustaining an erection. The sexual dysfunction can include erectile dysfunction (ED) and premature ejaculation (PE). The sexual dysfunction can be of physical or psychological origin. The origin of the physical component can be either neurogenic, i.e., caused by nerve damage, or vasculogenic, i.e., caused by arterial damage or other causes that are less common, i.e. hormonal, metabolic, or result from surgeries or medications. It is contemplated that subjects having sexual dysfunction of psychogenic origin may be benefited by the treatment because the confidence built up in having a satisfactory erection will eliminate future performance fears.

### Pharmaceutical Compositions

The method includes administration of a pharmaceutical composition of one or more agents effective to cause a male to sustain an erection. The compositions of the invention can be admixtures of one or more of agents known to produce an erection in a male subject. Any agent known to produce an erection in a male subject can be used. In preferred embodiments, the agent can be a vasodilator or a combination of vasodilators shown to be effective for treatment of erectile dysfunction.

Any vasodilator suitable for the purpose of producing an erection can be used. Many agents, with differing mechanisms of action, are known as vasodilators. In preferred embodiments, suitable vasodilators can include prostaglandin E1 (PGE1), papaverine, phentolamine, and atropine. The prostaglandins are a family of important regulatory hormone-like compounds found mainly in the genital organs. PGE1 is a vasodilator that acts as a potent smooth muscle relaxant by functioning as an alpha adrenergic blocker, as well as by elevating intracellular levels of cGMP. Papaverine acts as a smooth muscle relaxant by inhibiting cyclic mononucleotide phosphodiesterases, leading to increased cAMP and cGMP concentrations. It is known to block voltage dependent calcium channels. Phentolamine is known as an antihypertensive agent. It is a competitive, nonspecific alpha-adrenoceptor antagonist that works by direct action on smooth muscle to cause relaxation. Atropine is an anti-muscarinic anti-cholinergic drug believed to stimulate the release of nitrous oxide from the sinusoidal endothelium of corporal tissue. Other vasodilators and agents suitable for the purpose, acting by these or other mechanisms, may be determined and used alone or in combination using the disclosed methods.

The compositions used in the methods of the invention can be provided, e.g., to a clinic, in several base formulations that are further tailored to the individual needs of the patient by the methods described herein. The base formulations can include a single agent or a combination of agents. Any number of agents suitable for the purpose can be used in the combination. In formulations containing a combination of agents, the proportions of the particular agents within the combination can be varied. For ease of use by the practitioner, each base formulation can be designated by an identifier code number. Any number of base formulations can be provided, and any suitable means of identification can be used.

As an example of the method, seven base formulations can be provided, designated by identifier codes such as F0, F1, F2, F3, F4, F5 and F6. Formulation F0 can be, for example, a one-drug formulation, containing, e.g., PGE1 alone. Formulations may contain combinations of vasodilators, e.g., PGE1, papaverine, phentolamine, and atropine. In preferred embodiments, the formulations may include combinations containing all four of these drugs, or only three, i.e., papaverine, phentolamine, and atropine.

Each combination drug formulation can further be provided in multiple formulations that vary according to the relative proportions of the drugs within them. Availability of such premixed formulations provides a convenient means of prescribing amounts of the combined agents tailored in proportion to the particular needs of the patient. As an example, F1 can contain a mixture of stock solutions of PGE1, papaverine, phentolamine and atropine, combined by volume in the ratios of 15:45:25:15, respectively. F2 can contain the same four drugs, combined in the ratios of 30:35:20:15, respectively. F3 can contain these drugs in the ratios of 50:20:25:5, respectively. F5 can contain the four vasodilators in the same ratios as F2, but at double their concentrations. F6 is a diluted version of F2, containing the same four vasodilators at half strength. A three-drug formulation can be included, designated, e.g., F4, that contains a combination of three vasodilators, e.g., papaverine, phentolamine and atropine. In preferred embodiments, these three drugs can be combined in the ratios of 40:40:20 by volume. Further details of methods of preparing three- and four-drug formulations found to be effective in the practice of the invention are provided in the examples below.

All formulations can be provided in a suitable pharmaceutical vehicle. The components of the composition will preferably be in a vehicle that is physiologically compatible and that will allow the dissolution or suspension of the composition components. The components are preferably in an aqueous based pharmaceutically acceptable solution.

Suitable pharmaceutical vehicles are well known to those skilled in the art and are described in several manuals, including Remington's Pharmaceutical Sciences, 15th edition. When PGE1 is used without the other components, it is not stable in water at room temperature although for practical purposes it may be at room temperature for short periods of time. Similarly, formulations containing PGE1, papaverine, phentolamine and atropine, should be kept under refrigeration. A combination of papaverine, phentolamine and atropine can be stored without refrigeration.

### Assessment of Sexual Dysfunction

The invention includes a method of treating sexual dysfunction in a male mammal that includes ICP administration of a pharmaceutical composition of one or more agents in an amount effective to cause the male to sustain an erection. It has been shown that the most effective combinations of agents can be determined with the least number of test doses, and with minimization of unwanted side effects of incorrectly prescribed medication, by the disclosed methods.

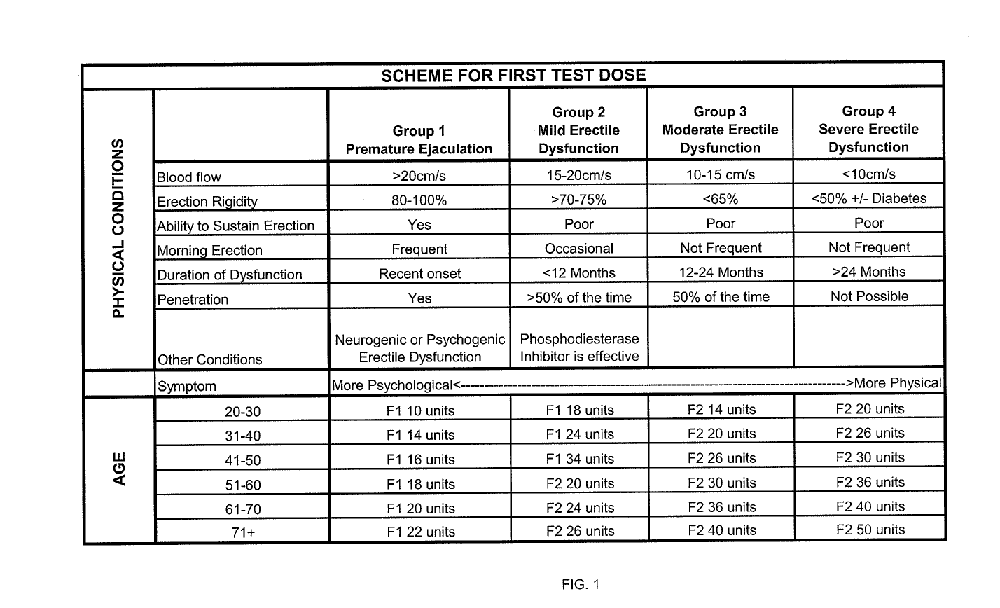

FIG. 1 is chart showing a scheme for determining a test dosage for patients with premature ejaculation or erectile dysfunction. In a first step in the method of treatment, subjects are assessed to determine the nature of their sexual dysfunction. In the practice of the invention with human males, patient health history and the nature of the sexual problem are determined by interviewing the subject. It is important that the medical history be thorough and detailed in order alert the practitioner to any possible medical conditions that should be addressed. Information obtained from the history can include significant medical conditions, e.g. diabetes, medications currently taken, use of tobacco or alcohol and drug abuse if any, as well as age and an overall assessment of health by the patient.

The subject is further asked to a respond to questions regarding a number of clinical indicators. The clinical indicators can include but are not limited to: the nature of the erection the patient is capable of sustaining during sexual encounters and by masturbation, the duration of the period of erectile dysfunction, the absence or presence and nature of the patient's morning erections, the percentage frequency of erections sufficient for vaginal penetration, the response of the patient to phosphodiesterase inhibitors, such as sildenafil, tadalafil, vardenafil, udenafil, and avanafil, if known, and the occurrence, if any, of premature ejaculation.

It is generally considered that 10 Newtons of force or almost 1000 g is required for an erected penis to penetrate vaginally with ease. In clinical practice, the rigidity of the penis can be measured using an Erectile Quality Monitor (EQM), a spring loaded device that measures the amount of force applied against it. The tip of the spring is a flat surface against which the head of an erected penis is pressed against until the penis is about to buckle or to bend. The maximum amount of force at the point of buckling can be read and expressed as a percentage of 1000 g. For example, if a penis exerts 700 g of force, then the penis is 70% erected.

### Formulation of Test Dose

The method includes a step of formulating a test dose of a pharmaceutical composition effective to cause the male to sustain an erection. Formulation of a diagnostic test dose of medication is guided by a compilation of clinical indicators. As stated above, the clinical indicators can be compiled such that patients can be assigned to one of several initial treatment groups. In some embodiments of the method, the clinical indicators can be displayed in the form of a chart that correlates the compiled clinical information with the selection of a test dose formulation.

Any number of treatment groups can be designated, and any combination of appropriate clinical indicators may be used. Determination of treatment group categories and selection of appropriate formulations is based on clinical experience with a population of subjects who fall into categories having particular combinations of indicators, including age groups. Compilation of such information from a population can be used to predict the appropriate effective combination and dosage of agents to be used in a customized formulation for a patient who falls into a particular treatment group.

In an example of this method, it has been determined that the longer the patient has had the erectile problem, the more likely it will be that he will require a stronger formula or a greater dose of formulations described herein. The rigidity and duration of a patient's erections is also informative: the less rigid and the less able to penetrate, the more advanced the problem, indicating the need to provide a stronger formula or a greater dose. If the patient has tried sildenafil, his response to this medication is instructional: failure to respond to sildenafil indicates a greater likelihood of requiring a higher dose or a stronger formula. Conversely, a good response to this drug tends to imply that a weaker dosage will be appropriate. The choice of test dosage can also be guided according to the nature of the patient's problem. For example, a patient seeking help for erectile dysfunction (ED) may require a higher dose or a stronger formula than a patient with a problem of premature ejaculation (PE). The quality of a patient's morning erections can also provide information about the nature of the problem. The stronger a patient's erections are in the mornings, the more likely it is that the problem is of psychological origin and hence can be resolved using a weaker dose. Age has also found to be a factor in patient response to a particular formulation: the older the patient, the less responsive he is to the medication. Therefore, a greater dose or stronger formulation is generally required with increasing patient age.

An example of the method for formulating the First Test Dose is shown in FIG. 1. In this example, compiled clinical indicators were used to define four categories of male erectile dysfunction (designated Groups 1-4), for purposes of choosing a test dose of medication. The choice of formulation of the test dose was further varied within each of the four groups according to the age of the patient.

Assessment of the subject can include the use of physical diagnostic tests which may further assist in determining the formulation of the test dose. A useful diagnostic test is a measurement of blood flow through the penis. Any method suitable for performing this measurement can be utilized. In a preferred embodiment of the method, this test is performed using Echo-Doppler. This test is used to determine if there has been any reduction in blood flow through the patient's cavernosal arteries. Blood flow can be used as a parameter for categorizing patients into diagnostic test groups. As an example, blood flow values may be divided into four groups, as shown in the chart in FIG. 1, e.g., with ranges of blood flows correlated with groupings, such as Group 1, >20 cm/s; Group 2, 15-20 cm/s; Group 3, 10-15 cm/s; Group 4, <10 cm/s.

A further physical diagnostic test that may be used is measurement of the sensitivity of the nerve system to the head of the penis. Any method suitable for performing this measurement can be used. A preferred method to perform this test is biothesiometry, in which the vibrating probe is placed at various parts of the penis and the patient is asked to report the presence of such vibration. The degree of vibration continues to be lowered until the patient can barely perceive its presence, at which point the lowest vibratory threshold is established and expressed in the units of voltage. For patients with ED, biothesiometry is used to determine if there is any neuropathy (nerve damage) which is self evident when the lowest vibratory threshold recorded is outside of the upper range of normal. Evidence of neuropathy without other contributory factors generally implies that a lower dosage or weaker formula is required for the First Test dose as seen in FIG. 1, under Other Conditions. For patients with Premature Ejaculation, biothesiometry is used to determine the degree of sensitivity of the glans or head of the penis. The lowest vibratory threshold is outside of the lower range of normal, The lower the voltage recorded, the more sensitive the penis is, the lower the dosage or the weaker the formula is required for the First Test Dose.

With reference to FIG. 1, the male is placed in one of four groups based on his physical condition and psychological symptoms. Group 1 is the premature ejaculation group, where the male has a blood flow rate of >20 cm/s, the rigidity of erection is 80-100%, has the ability to sustain an erection, has morning erection at least 2-3 times a week, has the ability to penetrate, where the duration of the dysfunction is recent, or the condition is neurogenic or psychogenic. Group 2 is the mild erectile dysfunction group, where the male has a blood flow rate of 15-20 cm/s, the rigidity of erection is 70-75%, has poor ability to sustain an erection, has occasional morning erections that occur less often, can penetrate over 50% of the time, has suffered the dysfunction for less than 12 months, or is responsive to phosphodiesterase inhibitors. Group 3 is the moderate erectile dysfunction group, where the male has a blood flow rate of 10-15 cm/s, the rigidity of erection is 65%, has poor ability to sustain an erection for vaginal penetration, has no or rarely occurring morning erections, can penetrate about 50% of the time, and has suffered the dysfunction between 12 to 24 months. Group 4 is the severe erectile dysfunction group, where the male has a blood flow rate of less than 10 cm/s, the rigidity of erection is less than 50%, he may suffer from diabetes, has no or rarely occurring morning erection, is unable to penetrate, and has suffered the dysfunction for over 24 months.

Nonetheless, these criteria are not group specific but rather indicative. i.e. these physical attributes help determine the grouping of erectile dysfunction and they tend to occur together as a compilation of signs which are then consistent with the physical manifestation of one's ability to achieve and maintain an erection. No single measure dictates the grouping. However, the dominant criterion in all of these physical conditions is the ability to “penetrate” regardless of all other conditions listed above it. In other words, the condition “Penetration” trumps other conditions, with the following two exceptions:

When VIAGRA® compound for treating erectile dysfunction made by Pfizer, Inc., LEVITRA® compound for treating erectile dysfunction made by Bayer Pharmaceuticals Corp., or CIALIS® compound for treating erectile dysfunction made by Eli Lilly and Company is effective in inducing an erection, in such case, this condition “trumps” all others and erectile dysfunction is classified as Group 2.

When the history points to the condition of neurogenic or psychogenic erectile dysfunction, the patient is assigned to Group 1.

Neurogenic is defined as a neurological condition known to cause or associate with erectile dysfunction such as Multiple Sclerosis, Parkinson's Disease, spinal cord lesions or trauma (such as recent spinal cord injury).

With respect to psychogenic, history suggests a patient's experiences performance anxiety described by physical signs such as palpitation, hyperventilation, sweating, feeling jittery or jumpy, sufficient to affect his confidence in love making. He can otherwise achieve full erections with masturbation, or experience night time erections and morning erections. This diagnosis can also be given to men who can consistently (sexually) perform with one partner but not with another partner.

Regardless of “other” physical conditions, a diagnosis of neurogenic or psychogenic erectile dysfunction would place the patient in Group 1 as the first test dose requires minimal strength.

Based upon the combination of information gained from the interview and physical testing, an appropriate formulation is selected to be used in a test dose of medication. As described above, the amount and composition is based on the determined clinical indicators, as well as the age of the patient. In an embodiment using a chart for compilation of clinical indicators and suggested dosages, for example as shown in FIG. 1, the formulation and volume of the composition to be tested can be determined as indicated in the lower portion of the chart in FIG. 1. For example, a male with Premature Ejaculation (Group 1) between the age of 31-40 would be prescribed 14 units of F1 as the test dose; a male with Mild ED (Group 2) between the age of 41-50, would be prescribed 34 units of F1; a male with Moderate ED (Group 3) between the age of 51-60, would be prescribed 30 units of F2; and a male with Severe ED (Group 4) between the age of 61-70 would be prescribed 40 units of F2. Furthermore, if the male's medical history suggests a strong psychological component to the dysfunction, the decision of grouping would veer towards the left. On the other hand, if the dysfunction is more physical, the grouping would veer towards the right.

### Administration of Test Dose

The method according to the present invention includes a step of administering by a test dose of a pharmaceutical composition effective to cause a male to sustain an erection. The test dose of the medication is preferably administered intracavernosally, i.e., by ICP, because this method delivers the medication to its precise site of action (i.e., the corpus cavernosum), minimizing unwanted side effects caused by systemic absorption.

The method further includes a step of observing one or more characteristics of the erection in order to evaluate the quality of the erection that results from administration of the test dose. Any method suitable for evaluating the quality of the erection can be used. Suitable methods can include palpation, assessing the rigidity or hardness of the penis as reported by the patient or objectively measured using an EQM, timing of initiation and duration of erection following administration of the test dose, ability to penetrate, presence or absence of discomfort, erectile response to erotic stimulus, and time required for return of flaccidity. Yet another suitable test is to measure the change in blood flow to the penis following administration of the first test dose using Echo-Doppler. Once the response to the test formulation has been determined, an adjustment can be made depending on this response. The dosage can be either reduced or increased or a new formula can be used, depending on the quality of the erection that the patient has achieved using the diagnostic dose. In some embodiments, directions for adjustment of the dosage can be compiled into a table, as well as directions for prescribing a dosage based on the results of the diagnostic test step.

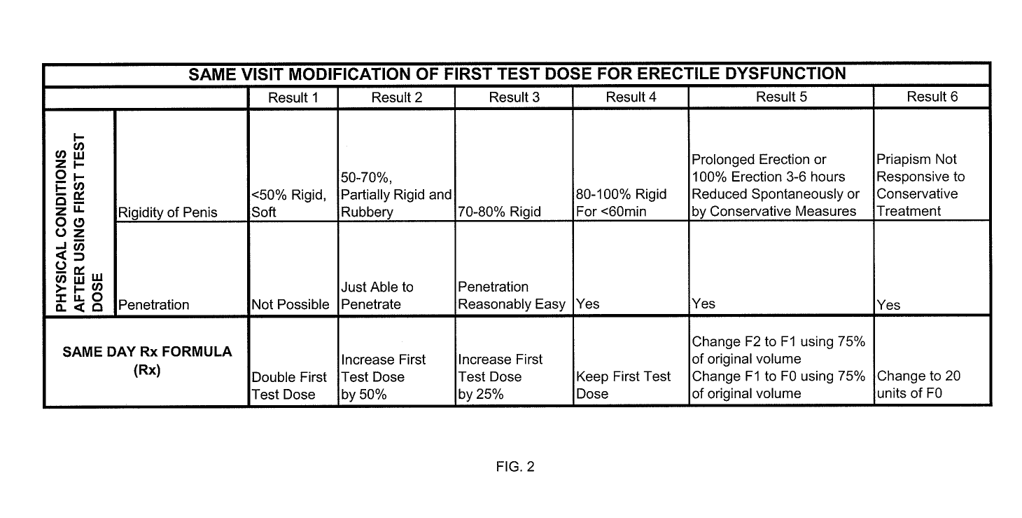

FIG. 2 is a chart illustrating a portion of the inventive method involving same visit modification of the first test dose to produce a same-visit prescription (Rx) for erectile dysfunction. In accordance to an embodiment of the present invention, the physical manifestation of the penis as a result of the test dose for the treatment of erectile dysfunction is observed and the male is placed in one of six result groups. Then, in accordance with the present invention, a same day prescription (Rx) would be prescribed based on the results. As shown in FIG. 2, for Result 1, the penis has rigidity less than 50%, soft, and the male is unable to penetrate. Therefore, the original dosage would be doubled, i.e. for a male taking 20 units of F2, such that the same visit modification of prescription (Rx) would be 40 units of F2. For Result 2, the penis is between 50-70%, partially rigid and rubbery, and the male is just able to penetrate. Therefore, the original test dosage is increased by 50%. For Result 3, the penis is 70-80% rigid and the male can penetrate reasonably easy. Therefore, the original test dose is increased by 25%. For Result 4, the penis is 80-100% rigid for less than 60 minutes and the male can penetrate. Therefore, the original test dose is used as the prescription dose. For Result 5, the male experiences prolonged erection or 100% erection for 3-6 hours and can penetrate. But the erection becomes detumescent or subsides over a period of 3-6 hours or by conservative measure. For example, by taking pseudoephedrine, taking a warm bath, sitting in a hot tub, or exercising. Therefore, the modified dose would be changed from F2 to F1 or from F1 to F0 using 75% of the original volume. For example, if the test dose was 30 units of F2, the modified prescription (Rx) would be 75% of 30 units of F1, i.e. 22.5 units of F1; if the test dose was 24 units of F1, the modified prescription (Rx) would be 75% of 24 units of F0, i.e. 18 units of F0. For Result 6, the male can penetrate and has a priapism that is not responsive to conservative treatment and the priapism has to be reduced by intracorporal injection of phenylephedrine or epinephrine or aspiration/drainage. Therefore, the modified prescription (Rx) would be 20 units of F0.

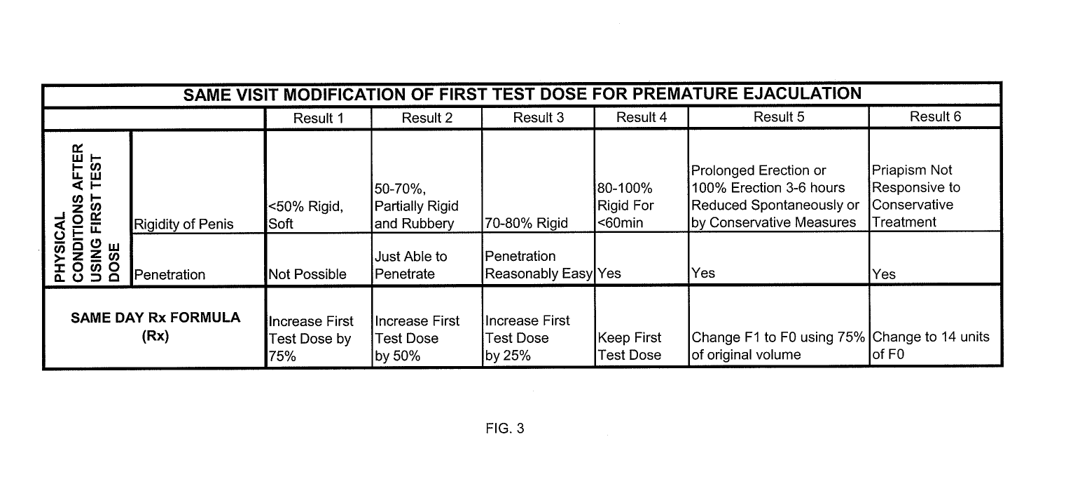

FIG. 3 is a chart illustrating a portion of the inventive method involving same visit modification of the first test dose to produce a prescription (Rx) for premature ejaculation. In accordance to an embodiment of the present invention, the physical manifestation of the penis as a result of the test dose for the treatment of premature ejaculation is observed. Similar to FIG. 2 for the treatment for erectile dysfunction, the male is placed in one of six result groups. Then, in accordance with the present invention, a same day prescription (Rx) would be prescribed based on the results. As shown in FIG. 3, for Result 1, the penis has rigidity less than 50%, soft, and the male is unable to penetrate. Therefore, the original dosage would be increased by 75%, i.e. for a male taking 20 units of F1, the same visit modification of prescription (Rx) would be 35 units of F1. For Result 2, the penis is between 50-70%, partially rigid and rubbery, and the male is just able to penetrate. Therefore, the original test dosage is increased by 50%. For Result 3, the penis is 70-80% rigid and the male can penetrate reasonably easy. Therefore, the original test dose is increased by 25%. For Result 4, the penis is 80-100% rigid for less than 60 minutes and the male can penetrate. Therefore, the original test dose is used as the prescription dose (Rx). For Result 5, the male experiences prolonged erection or 100% erection for 3-6 hours and can penetrate. But the erection becomes detumescent or subside over a period of 3-6 hours or by conservative measures such as taking pseudoephedrine, taking a warm bath, sitting in a hot tub, or exercising. Therefore, the modified dose would be changed from F0 to F0 using 75% of the original volume. For example, if the test dose was 24 units of F1, the modified prescription (Rx) would be 75% of 24 units of F0, i.e. 18 units of F0. For Result 6, the male can penetrate and has a priapism that is not responsive to conservative treatment and the priapism has to be reduced by intracorporal injection of phenylephedrine or epinephrine or aspiration/drainage. Therefore, the modified prescription (Rx) would be 14 units of F0.

In another aspect, the invention includes a method of determining an effective dose of a composition including one or more agents for treating erectile dysfunction in a male. The method includes the steps of a) assessing the general, physical, and psychological condition of the male; b) administering a test dose guided by the assessment performed in step a); c) observing at least one erection characteristic of the male after administration of the test dose; and d) adjusting the composition to provide an effective erection.

### Adjustment For Erectile Dysfunction

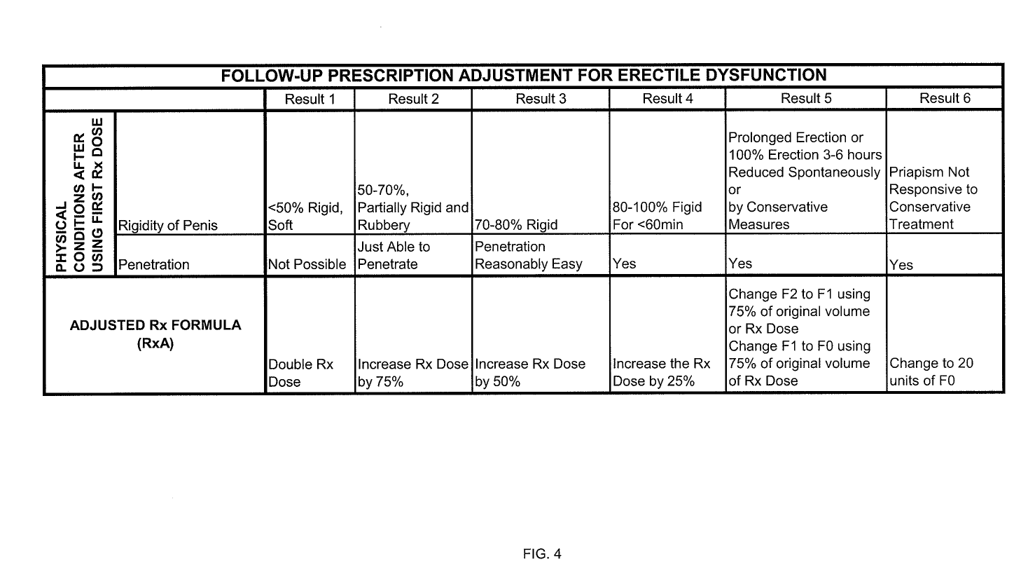

FIG. 4 is a chart illustrating a portion of the inventive method involving prescription adjustment for erectile dysfunction. Because individual results may vary, the same day modified prescription (Rx) may need fine tuning to address other unknown factors that contribute to the dysfunction. For example, home environment, physical attraction of the partner, other psychological tributes to the dysfunction, etc. According to an embodiment of the present invention, FIG. 4 demonstrates an adjustment of prescription (RxA) for erectile dysfunction.

A male's physical condition after using the first Rx dose is categorized in six result groups and each has a corresponding adjustment. As shown in FIG. 4, for Result 1, the penis has rigidity less than 50%, soft, and the male is unable to penetrate. Therefore, the first Rx dosage would be doubled, i.e. for a male taking 30 units of F2, the adjusted prescription (RxA) would be 60 units of F2. For Result 2, the penis is between 50-70%, partially rigid and rubbery, and the male is just able to penetrate. Therefore, the first Rx dosage (Rx) is increased by 75%. For Result 3, the penis is 70-80% rigid and the male can penetrate reasonably easy. Therefore, the first Rx dosage (Rx) is increased by 50%. For Result 4, the penis is 80-100% rigid for less than 60 minutes and the male can penetrate. Therefore, the first Rx dosage (Rx) is increased by 25%. For Result 5, the male experiences prolonged erection or 100% erection for 3-6 hours and can penetrate. But the erection becomes detumescent or subsides over a period of 3-6 hours or by conservative measures such as taking pseudoephedrine, taking a warm bath, sitting in a hot tub, or exercising. Therefore, the adjusted prescription (RxA) would be changed from F2 to F0 using 75% of the first Rx dosage (Rx) and F0 to F0 using 75% of the first Rx dosage (Rx). For example, if the modified prescription (Rx) was 24 units of F1, the adjusted prescription (RxA) would be 75% of 24 units of F0, i.e. 18 units of F0. For Result 6, the male can penetrate and has a priapism that is not responsive to conservative treatment and the priapism has to be reduced by intracorporal injection of phenylephedrine or epinephrine or aspiration/drainage. Therefore, the adjusted prescription (RxA) would be 20 units of F0.

### Resistant Erectile Dysfunction

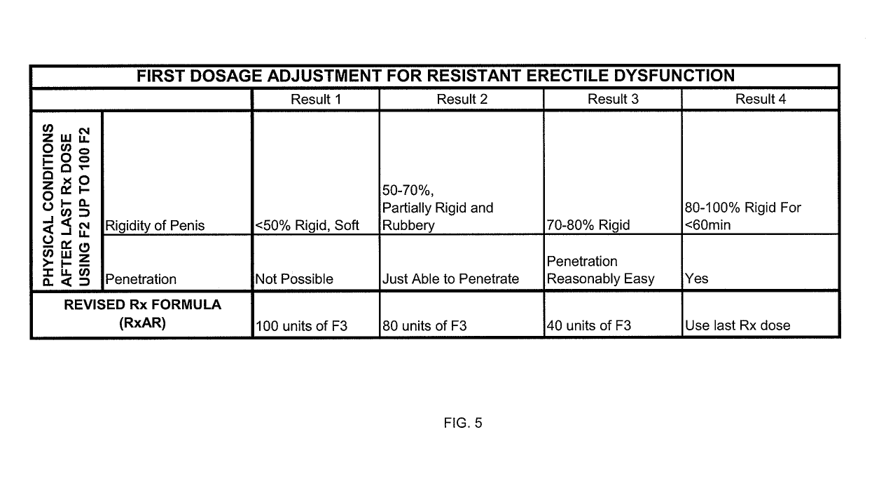

It has been found that over 95% of erectile dysfunction patients would respond positively to F2. However, in advanced cases, for example significant damage or fibrosis, the standard F2 does not work, even at 100 units. For example, a 45 year old patient in Group 4 (Severe Erectile Dysfunction) would be given a 30 units of F2 as the first test dose. During the same visit modification of the first test dose, if the patent is determined to be in Result group 1, the first test dose of 30 units of F2 would be doubled, resulting in 60 units of F2. If the patent is unsatisfied with the result of the 60 units of F2, and the prescription can be subsequently increased during the follow-up visits. However, if the patient is not responsive to the treatment of 100 units of F2, a more potent formula F3 should be administered. According to an embodiment of the present invention, FIG. 5 is a chart showing a scheme for a first adjustment for resistant erectile dysfunction.

A male's physical condition after using the adjusted prescription (RxA) is categorized in four result groups and each has a corresponding adjustment. As shown in FIG. 5, for Result 1, the penis has rigidity less than 50%, soft, and the male is unable to penetrate. The adjusted resistant erectile dysfunction Rx dosage (RxAR) would be 100 units of F3. For Result 2, the penis is between 50-70%, partially rigid and rubbery, and the male is just able to penetrate. Therefore, the adjusted resistant erectile dysfunction Rx dosage (RxAR) is 80 units of F3. For Result 3, the penis is 70-80% rigid and the male can penetrate reasonably easy. Therefore, the adjusted resistant erectile dysfunction Rx dosage (RxAR) is 40 units of F3. For Result 4, the penis is 80-100% rigid for less than 60 minutes and the male can penetrate. Therefore, the adjusted resistant erectile dysfunction Rx dosage (RxAR) is the same as the previous dosage.

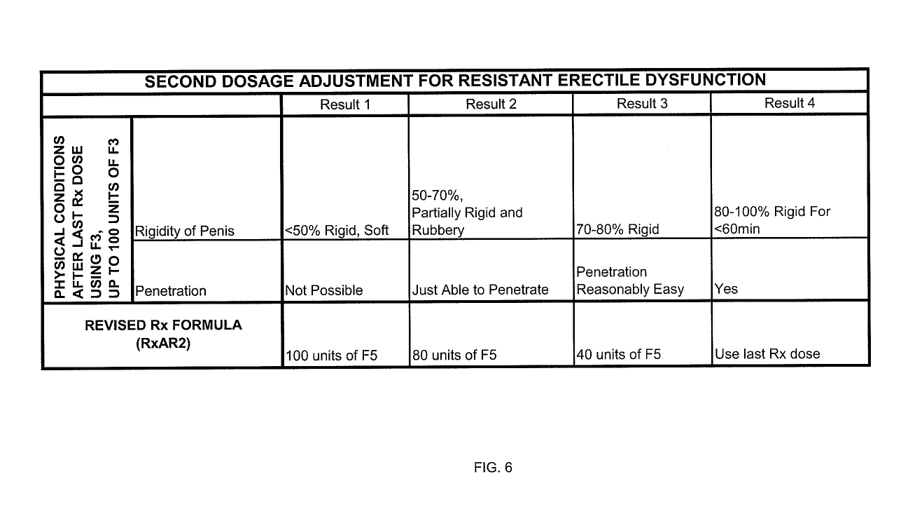

If the male still cannot successfully achieve erection after administration of 100 units of F3, a second adjustment for resistant erectile dysfunction is necessary. In this situation, the male will be administered with F5. FIG. 6 is a chart illustrating a portion of the inventive method involving a second adjustment for resistant erectile dysfunction. A male's physical condition after using the adjusted resistant erectile dysfunction Rx dosage (RxAR) is categorized in four result groups and each has a corresponding adjustment. As shown in FIG. 6, for Result 1, the penis has rigidity less than 50%, soft, and the male is unable to penetrate. The second adjusted resistant erectile dysfunction Rx dosage (RxAR2) would be 100 units of F5. For Result 2, the penis is between 50-70%, partially rigid and rubbery, and the male is just able to penetrate. Therefore, the second adjusted resistant erectile dysfunction Rx dosage (RxAR2) is 80 units of F5. For Result 3, the penis is 70-80% rigid and the male can penetrate reasonably easy. Therefore, the second adjusted resistant erectile dysfunction Rx dosage (RxAR2) is 40 units of F5. For Result 4, the penis is 80-100% rigid for less than 60 minutes and the male can penetrate. Therefore, the second adjusted resistant erectile dysfunction Rx dosage (RxAR2) is the same as the adjusted resistant erectile dysfunction Rx dosage (RxAR).

### Adjustment For Premature Ejaculation

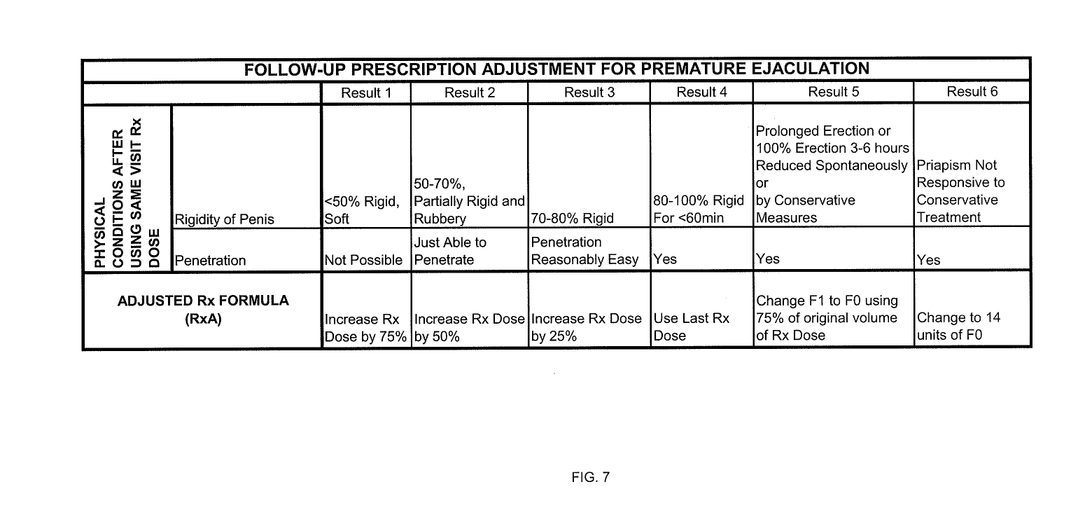

FIG. 7 is a chart showing a scheme for prescription adjustment for premature ejaculation. Because individual results may vary, the same visit prescription (Rx), see FIG. 3, may need fine tuning to address other unknown factors that contribute to the dysfunction.

A male's physical condition after using the first Rx dose is categorized in six result groups and each has a corresponding adjustment. As shown in FIG. 7, for Result 1, the penis has rigidity less than 50%, soft, and the male is unable to penetrate. Therefore, the first Rx dosage would be increased by 75%, i.e. for a male taking 20 units of F1, the adjusted prescription (RxA) would be 35 units of F1. For Result 2, the penis has rigidity between 50-70%, partially rigid and rubbery, and the male is just able to penetrate. Therefore, the first Rx dosage (Rx) is increased by 50% to result the adjusted prescription (RxA). For Result 3, the penis has rigidity of 70-80% and the male can penetrate reasonably easy. Therefore, the first Rx dosage (Rx) is increased by 25% to result the adjusted prescription (RxA). For Result 4, the penis has rigidity of 80-100% for less than 60 minutes and the male can penetrate. Therefore, the dosage (RxA) would remain the same as the last Rx dose. For Result 5, the male experiences prolonged erection or 100% erection for 3-6 hours and can penetrate, but the erection becomes detumescent or subsides over a period of 3-6 hours or by conservative measures such as taking pseudoephedrine, taking a warm bath, sitting in a hot tub, or exercising. Therefore, the adjusted prescription (RxA) would be changed from F0 to F0 using 75% of the first Rx dosage (Rx). For example, if the prescription (Rx) was 24 units of F1, the adjusted prescription (RxA) would be 75% of 24 units of F0, i.e. 18 units of F0. For Result 6, the male can penetrate and has a priapism that is not responsive to conservative treatment and the priapism has to be reduced by intracorporal injection of phenylephedrine or epinephrine or aspiration/drainage. Therefore, the adjusted prescription (RxA) would be 14 units of F0.

Conversion from Refrigerated to Non-Refrigerated Formula

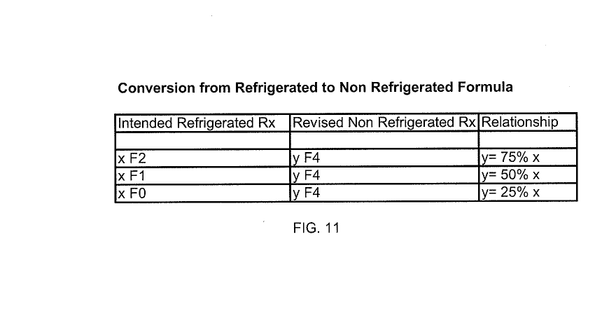

As discussed above, F0 contains PGE, and F1, F2, F3, F5, and F6 contain different ratios of PGE1, papaverine, phentolamine, and atropine. These formulae require refrigeration to maximize shelf life. Some patients prefer a non-refrigerated formula for ease of transportation, and F4 is introduced. First, the patient would be diagnosed and given either a prescription of F0 or F1 or F2. Upon the patient's request, the physician can convert the intended Rx of F0 or F1 or F2 to F4. FIG. 11 is a chart illustrating a portion of the inventive method involving a non-refrigerated formula. As shown in FIG. 11, if the patient is prescribed with X units of F0, then the dosage for F4 would be 25% of X. For example, if the patient is prescribed with 100 units of F0, then the prescription for F4 would be 25 units. Along the same line, if the patient is prescribed with X units of F1, then the dosage for F4 would be 50% of X. For example, if the patient is prescribed with 50 units of F1, then the prescription for F4 would be 25 units. Similarly, if the patient is prescribed with X units of F2, then the dosage for F4 would be 75% of X. For example, if the patient is prescribed with 100 units of F2, the corresponding prescription for F4 would be 75 units.

### Benefits of Formula F6

At half the strength of F2, formula F6 is best used when a low dosage of F2 is prescribed, which can then be converted to F6 at twice the volume. For example, an Rx of 14 units of F2 can be converted to 28 units of F6. It is found that a lower dosage can be difficult for an older patient to draw out of the vial himself and can be more difficult to inject. Further, the higher volume of F6 would result in more uniform erectile response and reduce the risk of localized fibrosis.

### Treatment of Sexual Dysfunction in a Population

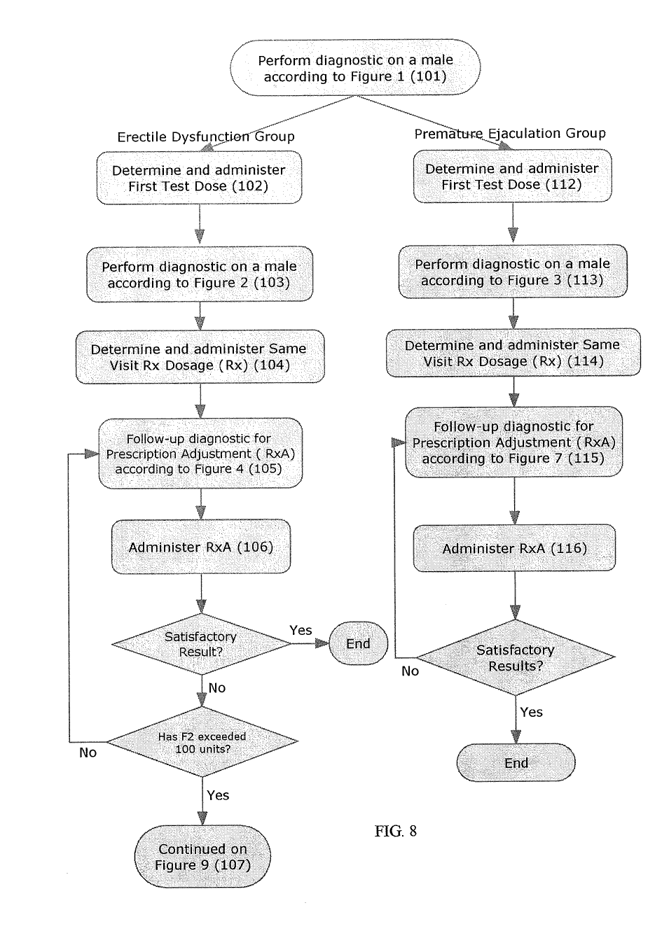

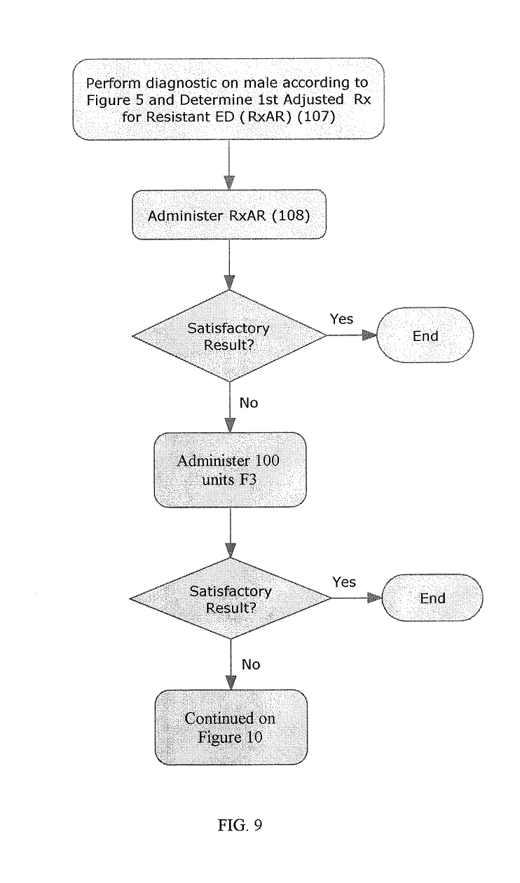

An another embodiment of the invention further provides a method for a same visit treatment and follow-up treatments of achieving and maintaining male erectile response regardless of state of mind, occurrence of ejaculation, or lack of sexual arousal of a patient. FIGS. 8 and 9 are flow diagrams illustrating the steps of the method using the charts in FIGS. 1-7. These flow diagrams illustrate how a doctor can provide a comprehensive and extended diagnostic and treatment method according to an embodiment of the present invention on the patient. In step 101, the doctor performs an initial diagnostic on the patient by making observations on the physical and psychological conditions of the patient according to the inventive method as partially illustrated in the chart in FIG. 1. Based on the physical and psychological conditions of the patient, the patient is identified to be either in Group 1 (Premature Ejaculation), Group 2 (Mild Erectile Dysfunction), Group 3 (Moderate Erectile Dysfunction), or Group 4 (Severe Erectile Dysfunction). If the patient is in Groups 2-4, the doctor follows step 102; determines the patient's age and provides him a first test dose. However, if the patient is in Group 1, the doctor will follow step 112; determine the patient's age and provide him with a first test dose.

For patients having erectile dysfunction, upon receiving the first test dose, the patient's reaction to the first test dose is observed during the visit. Then, in step 103, the doctor performs a diagnostic on the patient according to FIG. 2. Next, in step 104 the doctor prescribes a same visit prescription dosage (Rx) to the patient. The treatment is then self-administered by the patient.

As stated above, due to environmental or psychological factors, follow-up visits might be necessary. In step 105, the doctor performs another diagnostic on the patient according to FIG. 4 and depending on the patient's condition, a follow-up adjustment dosage (RxA) is provided to the patient. In step 106, RxA is administered to the patient. If the patient experiences a satisfactory result, the process ends. However, if the patient is not satisfied with the result and the patient has been administered less than 100 units of F2, steps 105 and 106 are repeated, where in step 105, the dose adjustment applied according to FIG. 4 is made to the last RxA follow-up adjustment dosage, rather than the initial Rx dosage. This process continues until the patient is unable to sustain an erection using up to 100 units of F2.

In the event that the patient is unable to sustain an erection after using 100 units of F2, the doctor can switch the prescription to a more potent F3 composition for patients with resistant erectile dysfunction. Turning to FIG. 9, in step 107, the doctor performs another diagnostic on the patient according to FIG. 5 and determines the first adjusted prescription for resistant erectile dysfunction (RxAR) for the patient. In step 108, RxAR is administered to the patient. If the patient experiences a satisfactory result, the process ends. However, if the patient is not satisfy with the result, the patient is administered 100 units of F3. For example, if a patient in Result Group 3 using 40 units of F3 is unsatisfied with the result, his new adjusted dosage would be 100 units of F3. If the result is still not satisfactory, the physician shall retest the patient with 100 F3 and use penile ring, which works like a constriction device to limit the amount of venous leakage.

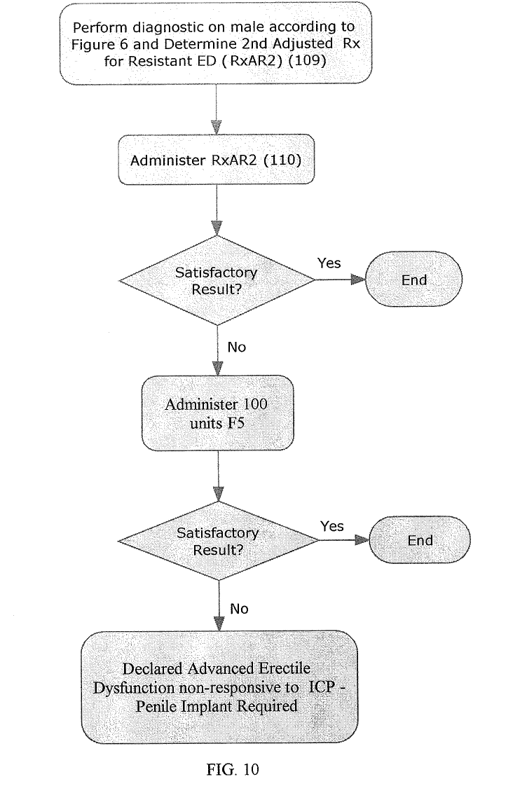

If the patient is unable to sustain an erection after using 100 units of F3, the doctor can switch the prescription to F5 for the patient. Turning to FIG. 10, in step 109, the doctor performs another diagnostic on the patient according to FIG. 6 and determines a second adjusted prescription (RxAR2) for resistant erectile dysfunction for the patient. In step 110, RxAR2 is administered to the patient. If the patient experiences a satisfactory result, the process ends. However, if the patient cannot sustain an erection, 100 units of F5 is administered. If 100 units of F5 fails, the patient is declared advanced erectile dysfunction non responsive to ICP—penile implant is required.

Returning to patients initially diagnosed with premature ejaculation, as shown in FIG. 8, in step 113, the patient's reaction to the first test dose is observed during the initial visit according to FIG. 3. Next, the doctor determines and prescribes a same visit dosage (Rx) to the patient in step 114. The treatment is then self-administered by the patient.

In step 115, the doctor performs another diagnostic on the patient according to FIG. 7 and, depending on the patient's condition, a follow-up adjustment dosage (RxA) is provided to the patient. In step 116, RxA is administered to the patient. If the patient experiences a satisfactory result, the process ends. However, if the patient cannot sustain an erection after the administration of RxA, steps 115 and 116 are repeated until a satisfactory result is achieved which it would eventually. The rationale for this progressive adjustment is to avoid the development of a priapism.

### Kits

The invention further includes kits containing the prescribed composition and an apparatus used by the patient to administer the composition. In a preferred embodiment, the apparatus can be a specialized syringe with an automatic injector. Such devices are known in the art of urology. The apparatus for self-injection can include an applicator into which a preloaded syringe is placed. The applicator can be designed such that with the press of a button, a fine needle is injected to the correct depth into the corpus cavernosum. This type of apparatus is advantageous both for its ability to regulate the depth of the injection, and for facilitating self-use by the subject. Also contemplated are home and travel kits that include individualized dosages of the customized composition, an apparatus for self-injection of the composition, and instructions for use.

In some embodiments, instructions included in the kit can be in the form of an animated videotape. The animations in the videotape can illustrate the correct method of holding the penis and the applicator during injection, the range of possible sites for injection and the procedures for dispensing medication from the injector and distributing the medication by massage of the penis after injection. The videotape can also include reminders to the patient about storage of the medication and information about how to obtain medical support regarding use of the medication. Inclusion of a videotape in the kit provides a useful and reassuring review for the patient of procedures discussed and demonstrated during visits to the clinic.

There is virtually no discomfort when the patient injects the composition because the central part of the penis is relatively insensitive in contrast to the penis head, which is a sensitive area. The central shaft is composed of spongy tissue and injection into this area places the solution into the cavernosa. The patient may have some initial trepidation concerning the first self-injection. This is readily overcome by instructions and practice in the physician's office and a complete instruction sheet or videotape accompanying the dosage kits. Fear of pain is dispelled, and in fact some patients need to be warned that a lack of pain is not an indication that the drug did not reach the targeted area.

In practice, the patient simply injects the proper dose into the proximal third of the penis. Typically, a rapid response is obtained within 5 minutes. The duration of the erection is, on average, 30 to 60 minutes. The penis remains erect during this period regardless of ejaculation, state of mind, or lack of sexual arousal, in contrast to agents such as sildenafil, with which the penis becomes flaccid after ejaculation. Erotic stimulation is not required with this treatment, as it is with sildenafil.

### EXAMPLES

### Example 1

### Preparation of Formulations for Erectile Dysfunction

This example describes the preparation of agents found to be effective and convenient for use in the methods of the invention, with designations corresponding to those indicated in the charts in FIGS. 1-7. In this example, six base formulations of the compositions were prepared and designated by code numbers F0, F1, F2, F3, F4, F5, and F6. Formulation F0 was prepared to include PGE1 only, at a concentration of 10 μg/ml. Formulations designated F1, F2, F3, F5 and F6 all contained combinations of four vasodilators, i.e., PGE1, papaverine, phentolamine, and atropine, but in differing proportions or concentrations. F1 contained a mixture of solutions of PGE1, papaverine, phentolamine and atropine, combined in the respective ratios of 15:45:25:15 by volume. The final formulations were achieved conveniently by combining mixtures of stock solutions in varying volume proportions. Specifically, a solution of PGE1 was prepared in saline at a concentration of 20 μg/ml. A solution of papaverine was prepared in a hydrochloride vehicle at a concentration of 30 mg/ml. Phentolamine was prepared in a hydrochloride solution at a concentration of 10 mg/ml, and atropine was prepared in a sulphate solution at a concentration of 1 mg/ml. Using solutions thus prepared, formulation F1, having the ratio of 15:45:25:15, was prepared by mixing 15 mls of the PGE1 (20 .mu.g/ml), 45 mls of papaverine (30 mg/ml), 25 mls of phentolamine (10 mg/ml) and 15 mls of atropine (1 mg/ml), for a total volume of 100 mls. Accordingly, final concentrations of the four vasodilators in formulation F0 were PGE1: 3 μg/ml; papaverine: 13.5 mg/ml; phentolamine: 2.5 mg/ml; and atropine: 0.15 mg/ml.

Formulation F2 contained the same four drugs, prepared from stock solutions at the concentrations stated above, but combined in the ratios of 30:35:20:15 by volume. Therefore the resulting formulation contained PGE1, papaverine, phentolamine, and atropine at the respective final concentrations of 6 μg/ml, 10.5 mg/ml, 2.0 mg/ml and 0.15 mg/ml. F3 contained the same four drugs combined in the ratios of 50:20:25:5, to yield a composition containing PGE1, papaverine, phentolamine, and atropine at the respective final concentrations of 10 μg/ml, 6.0 mg/ml, 2.5 mg/ml and 0.05 mg/ml. F5 contained the four vasodilators in the same ratios as F2, but at double their concentrations to yield concentrations of 12 μg/ml of PGE1, 21 mg/ml of papaverine, 4.0 mg/ml of phentolamine and 0.30 mg/ml of atropine. F6 was prepared by combining the same four vasodilators in the ratios for F2, but at half strength to yield concentrations of 3 μg/ml, 5.25 mg/ml, 1.0 mg/ml and 0.075 mg/ml.

Formulation F4 contained a combination of only three vasodilators, i.e., papaverine, phentolamine and atropine in the ratios of 40:40:20 by volume, using stock solutions prepared as described above. Final concentrations of papaverine, phentolamine and atropine solutions in formulation F4 were therefore 12.0 mg/ml, 4 mg/ml, and 0.2 mg/ml, respectively.

### Example 2

### Determination of Test and Effective Doses of Formulations

This example describes how test doses and adjusted doses of formulations designated by identifiers F0-F6, described herein, can be determined by a practitioner, by reference to guidelines provided in chart form. In the charts shown in this example (FIGS. 1-9), predictive clinical indicators were compiled into four patient groups having the observed combination of parameters. Correlations were made with dosages found to be effective for each of these groups. Referring to FIG. 1, it can be seen that to determine an appropriate test dose for a 30-year-old classified in Group 3, a suggested test dose is 14 units (0.14 ml) of formulation F2. By contrast, a 61-70 year old subject in the same group could initially be administered 36 units (0.36 ml) of formulation F2. In this example, all dosage volumes are shown in units, where 10 units=0.1 ml.

Referring now to FIG. 2, if the resulting erection is 70-80% in response to the test dose, the prescribed dose is increased by 25% of the volume used for the test dose (e.g., by 0.09 ml of F2 for a 61-70 year old subject with erectile dysfunction).

FIG. 2 provides additional guidance for selection of an effective dosage following administration of the test dose. For example, if the patient is in result group 5 or 6, formulations can be modified for the rare patients who experience a priapism or prolonged erection (3-6 hours) following administration of the test dose. If the problem resolves with conservative treatment or with the administration of an antidote such as epinephrine, as in Result Group 5, the same day Rx formulation is achieved by changing the test formulation from F2 to F1 (if the first test dose was F2), or F0 to F0 (if the first test dose was F0) using 75% of the original volume. If the patient experiences a priapism that does not resolve with conservative measures but requires draining, as in Result Group 6, the formulation is changed to F0 (containing PGE1 alone), starting at 20 units.

Also shown in FIG. 5 is direction for a practitioner whose patient experiences no response with the maximum dosage (100 units) of formulation F2. In this case, the patient can be tested with formula F3, which contains the same ingredients as F2 but in the ratios of 50:20:25:5, to yield a composition containing PGE1, papaverine, phentolamine, and atropine at the respective final concentrations of 10 μg/ml, 6.0 mg/ml, 2.5 mg/ml and 0.05 mg/ml. The dosage of the Revised Rx Formula depends on the Result Group and can be 100 units of F3 for result group 1, 80 units of F3 for result group 2, 40 units of F3 for result group 3, or the same as last Rx dose for result group 4.

The prescribed dosage table (Chart 1) also contains columns to separate patients with erectile dysfunction (ED) and those with premature ejaculation (PE). The reason for this separation is that patients with PE tend to require a more conservative first test dose than those with ED.

### Other Embodiments

While the methods and compositions of this invention have been described in terms of preferred embodiments, it will be apparent to those of skill in the art that variations may be applied to the methods and compositions, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for a same visit treatment of achieving and maintaining male erectile response regardless of state of mind, occurrence of ejaculation, or lack of sexual arousal of a patient, the method comprising:
(a) performing a diagnostic on the patient using physical conditions of the patient, selected from the group consisting of blood flow rate, rigidity of erection, a sustainability of an erection, occurrence of morning erection, duration of erectile dysfunction, or an ability to penetrate;
(b) determining an initial diagnostic group of the patient, selected from the group consisting of a premature ejaculation group, a mild erectile dysfunction, a moderate erectile dysfunction group, and a severe erectile dysfunction, wherein each diagnostic group has corresponding conditions to the physical conditions of the patient;
(c) identifying an age group of the patient;
(d) selecting a first test dose of a pharmaceutical composition, which corresponds to the age group and the diagnostic group of the patient, wherein the pharmaceutical composition is a solution including PGE1, papaverine, phentolamine, and atropine;
(e) administrating the first test dose of the pharmaceutical composition to the patient;
(f) observing the physical conditions of the patient after the administration of the first test dose, including the rigidness of the penis of the patient or whether the patient is able to penetrate;
(g) determining a result group of the patient after observing the physical conditions of the patient; and
(h) prescribing a same visit dosage for the patient based on the result group.

2. The method of claim 1, wherein the premature ejaculation group includes physical conditions wherein the patient's blood flow rate is >20 cm/s, the rigidity of erection is 80-100%, the sustainability of an erection is good, the occurrence of morning erection frequent, the duration of erectile dysfunction is recent onset, and the ability to penetrate is able.

3. The method of claim 1, wherein the mild erectile dysfunction group includes physical conditions wherein the patient's blood flow rate is about 15-20 cm/s, the rigidity of erection is about >70-75%; the sustainability of an erection is poor, the occurrence of morning erection occasional, the duration of erectile dysfunction is less than 12 months, and the ability to penetrate is greater than 50% of the time.

4. The method of claim 1, wherein the moderate erectile dysfunction group includes physical conditions wherein the patient's blood flow rate is about 10-15 cm/s, the rigidity of erection is less than 65%, the sustainability of an erection is poor, the occurrence of morning erection not frequent, the duration of erectile dysfunction between about 12 to about 24 months, and the ability to penetrate is about 50% of the time.

5. The method of claim 1, wherein the severe erectile dysfunction group includes physical conditions wherein the patient's blood flow rate is >10 cm/s, the rigidity of erection is less than 50%, the sustainability of an erection is poor, the occurrence of morning erection not frequent, the duration of erectile dysfunction is over 24 months, and the ability to penetrate is not possible.

6. The method of claim 1, wherein if the initial diagnostic group is mild erectile dysfunction, moderate erectile dysfunction, or severe erectile dysfunction, the result group includes:
a result group 1, wherein the patient's penis is less than 50% rigid and the patient is unable to penetrate;
a result group 2, wherein the patient's penis is 50-70%, partially rigid and rubbery and the patient is just able to penetrate;
a result group 3, wherein the patient's penis is 70-80% rigid and the patient can penetrate reasonably easy;
a result group 4, wherein the patient's penis is 80-100% rigid for less than 60 minutes and the patient can penetrate;
a result group 5, wherein the patient has prolonged erection or 100% erection for 3-6 hours, the erection is reduced spontaneously or by conservative measures, and the patient can penetrate; and
a result group 6, wherein the patient has priapism and is not responsive to conservative treatment and the patient can penetrate.

7. The method of claim 6, further comprising doubling the first test dose as the same visit dosage, for a patient in the result group 1.

8. The method of claim 6, further comprising increasing the first test dose by 50% as the same visit dosage, for a patient in the result group 2.

9. The method of claim 6, further comprising increasing the first test dose by 25% as the same visit dosage, for a patient in the result group 3.

10. The method of claim 6, further comprising keeping the first test dose as the same visit dosage, for a patient in the result group 4.

11. The method of claim 6, further comprising prescribing the same visit dosage by changing a prescription of a solution of PGE1, papaverine, phentolamine, and atropine in the ratio of 30:35:20:15, respectively to a solution in the ratio of 15:45:25:15, respectively and using 75% of the original volume as the same visit dosage, for a patient in the result group 5.

12. The method of claim 6, further comprising prescribing the same visit dosage by changing a prescription of a solution of PGE1, papaverine, phentolamine, and atropine in the ratio of 15:45:25:15, respectively to PGE1 and using 75% of the original volume as the same visit dosage, for a patient in the result group 5.

13. The method of claim 6, further comprising prescribing 20 units of PGE1 as the same visit dosage, for patient in the result group 6.

14. The method of claim 1, wherein if the initial diagnostic group is the premature ejaculation group, the result group includes:
a result group 1, wherein the patient's penis is less than 50% rigid and the patient is unable to penetrate;
a result group 2, wherein the patient's penis is 50-70%, partially rigid and rubbery and the patient is just able to penetrate;
a result group 3, wherein the patient's penis is 70-80% rigid and the patient can penetrate reasonably easy;
a result group 4, wherein the patient's penis is 80-100% rigid for less than 60 minutes and the patient can penetrate;
a result group 5, wherein the patient has prolonged erection or 100% erection for 3-6 hours, the erection is reduced spontaneously or by conservative measures, and the patient can penetrate; and
a result group 6, wherein the patient has priapism and is not responsive to conservative treatment and the patient can penetrate.

15. The method of claim 14, further comprising increasing the first test dose by 75% as the same visit dosage, for a patient in the result group 1.

16. The method of claim 14, further comprising increasing the first test dose by 50% as the same visit dosage, for a patient in the result group 2.

17. The method of claim 14, further comprising increasing the first test dose by 25% as the same visit dosage, for a patient in the result group 3.

18. The method of claim 14, further comprising keeping the first test dose as the same visit dosage as the same visit dosage, for a patient in the result group 4.

19. The method of claim 14, further prescribing the same visit dosage by changing a prescription of a solution of PGE1, papaverine, phentolamine, and atropine in the ratio of 15:45:25:15, respectively to PGE1 and using 75% of the original volume as the same visit dosage, for a patient in the result group 5.

20. The method of claim 14, further comprising prescribing 14 units of PGE1 as the same visit dosage, for a patient in the result group 6.

21. The method of claim 6, further comprising:
(i) observing the physical conditions of the patient after the administration of the same visit dosage, including the rigidness of the penis of the patient and whether the patient is able to penetrate;
(j) determining a follow-up visit result group of the patient for erectile dysfunction treatment after observing the physical conditions of the patient; and
(k) prescribing a follow-up adjustment dosage for the patient based on the follow-up result group.

22. The method of claim 21, wherein the follow-up result group includes:
a follow-up result group 1, wherein the patient's penis is less than 50% rigid and the patient is unable to penetrate;
a follow-up result group 2, wherein the patient's penis is 50-70%, partially rigid and rubbery and the patient is just able to penetrate;
a follow-up result group 3, wherein the patient's penis is 70-80% rigid and the patient can penetrate reasonably easy;
a follow-up result group 4, wherein the patient's penis is 80-100% rigid for less than 60 minutes and the patient can penetrate;
a follow-up result group 5, wherein the patient has prolonged erection or 100% erection for 3-6 hours, the erection is reduced spontaneously or by conservative measures, and the patient can penetrate; and
a follow-up result group 6, wherein the patient has priapism and is not responsive to conservative treatment and the patient can penetrate.

23. The method of claim 22, further comprising doubling the same visit dose as the follow-up adjustment dosage, for a patient in the follow-up result group 1.

24. The method of claim 22, further comprising increasing the same visit dose by 75% as the follow-up adjustment dosage, for a patient in the follow-up result group 2.

25. The method of claim 22, further comprising increasing the same visit dose by 50% as the follow-up adjustment dosage, for a patient in the follow-up result group 3.

26. The method of claim 22, further comprising increasing the same visit dose by 25% as the follow-up adjustment dosage, for a patient in the follow-up result group 4.

27. The method of claim 22, further comprising prescribing the follow-up dosage by changing a prescription of a solution of PGE1, papaverine, phentolamine, and atropine in the ratio of 30:35:20:15, respectively to a solution in the ratio of 15:45:25:15, respectively and using 75% of the original volume of the same visit dose as the follow-up adjustment dosage, for a patient in the follow-up result group 5.

28. The method of claim 22, further prescribing the follow-up dosage by changing a prescription of a solution of PGE1, papaverine, phentolamine, and atropine in the ratio of 15:45:25:15, respectively to PGE1 and using 75% of the original volume of the same visit dose as the follow-up adjustment dosage, for a patient in the follow-up result group 5.

29. The method of claim 22, further comprising prescribing 20 units of PGE1 as the follow-up adjustment dosage, for patient in the follow-up result group 6.

30. The method of claim 21, further comprising:
(l) observing the physical conditions of the patient after the administration of 100 units of a solution of PGE1, papaverine, phentolamine, and atropine in the amount of 6 μg/ml, 10.5 mg/ml, 2.0 mg/ml and 0.15 mg/ml, respectively, including the rigidness of the penis of the patient and whether the patient is able to penetrate;
(m) determining a resistant erectile dysfunction result group of the patient after observing the physical conditions of the patient; and
(n) prescribing a first adjustment dosage for resistant erectile dysfunction for the patient based on the resistant erectile dysfunction result group.

31. The method of claim 30, wherein the resistant erectile dysfunction result group includes:
a resistant erectile dysfunction result group 1, wherein the patient's penis is less than 50% rigid and the patient is unable to penetrate;
a resistant erectile dysfunction result group 2, wherein the patient's penis is 50-70%, partially rigid and rubbery and the patient is just able to penetrate;
a resistant erectile dysfunction result group 3, wherein the patient's penis is 70-80% rigid and the patient can penetrate reasonably easy; and
a resistant erectile dysfunction result group 4, wherein the patient's penis is 80-100% rigid for less than 60 minutes and the patient can penetrate.

32. The method of claim 31, further comprising prescribing 100 units of a solution of PGE1, papaverine, phentolamine, and atropine in the amount of 10 μg/ml, 6.0 mg/ml, 2.5 mg/ml and 0.05 mg/ml, respectively, as the first adjustment dosage for resistant erectile dysfunction, for a patient in the resistant erectile dysfunction result group 1.

33. The method of claim 31, further comprising prescribing 80 units of a solution of PGE1, papaverine, phentolamine, and atropine in the amount of 10 μg/ml, 6.0 mg/ml, 2.5 mg/ml and 0.05 mg/ml, respectively, as the first adjustment dosage for resistant erectile dysfunction, for a patient in the resistant erectile dysfunction result group 2.

34. The method of claim 31, further comprising prescribing 40 units of a solution of PGE1, papaverine, phentolamine, and atropine in the amount of 10 μg/ml, 6.0 mg/ml, 2.5 mg/ml and 0.05 mg/ml, respectively, as the first adjustment dosage for resistant erectile dysfunction for a patient in the resistant erectile dysfunction result group 3.

35. The method of claim 31, further comprising prescribing the same dosage as the first adjustment dosage for resistant erectile dysfunction for patient's in the resistant erectile dysfunction result group 4.

36. The method of claim 30, further comprising:
(o) observing the physical conditions of the patient after the administration of 100 units of a solution of PGE1, papaverine, phentolamine, and atropine in the amount of 10 μg/ml, 6.0 mg/ml, 2.5 mg/ml and 0.05 mg/ml, respectively, including the rigidness of the penis of the patient and whether the patient is able to penetrate;
(p) determining a second resistant erectile dysfunction result group of the patient after observing the physical conditions of the patient; and
(q) prescribing a second adjustment dosage for resistant erectile dysfunction for the patient based on the second resistant erectile dysfunction result group.

37. The method of claim 36, wherein the second resistant erectile dysfunction result group includes:
a second resistant erectile dysfunction result group 1, wherein the patient's penis is less than 50% rigid and the patient is unable to penetrate;
a second resistant erectile dysfunction result group 2, wherein the patient's penis is 50-70%, partially rigid and rubbery and the patient is just able to penetrate;
a second resistant erectile dysfunction result group 3, wherein the patient's penis is 70-80% rigid and the patient can penetrate reasonably easy; and
a second resistant erectile dysfunction result group 4, wherein the patient's penis is 80-100% rigid for less than 60 minutes and the patient can penetrate,

38. The method of claim 37, further comprising prescribing 100 units of a solution of PGE1, papaverine, phentolamine, and atropine in the amount of 12 μg/ml, 21 mg/ml, 4.0 mg/ml and 0.30 mg/ml, respectively, as the second adjustment dosage for resistant erectile dysfunction, for a patient in the second resistant erectile dysfunction result group 1.

39. The method of claim 37, further comprising prescribing 80 units of a solution of PGE1, papaverine, phentolamine, and atropine in the amount of 12 μg/ml, 21 mg/ml, 4.0 mg/ml and 0.30 mg/ml, respectively, as the second adjustment dosage for resistant erectile dysfunction, for a patient in the second resistant erectile dysfunction result group 2.

40. The method of claim 37, further comprising prescribing 40 units of a solution of PGE1, papaverine, phentolamine, and atropine in the amount of 12 μg/ml, 21 mg/ml, 4.0 mg/ml and 0.30 mg/ml, respectively, as the second adjustment dosage for resistant erectile dysfunction, for a patient in the second resistant erectile dysfunction result group 3.

41. The method of claim 37, further comprising prescribing the same dosage as the second adjustment dosage for resistant erectile dysfunction, for a patient in the second resistant erectile dysfunction result group 4.

42. The method of claim 14, further comprising:
(i) observing the physical conditions of the patient after the administration of the same visit dosage, including the rigidness of the penis of the patient and whether the patient is able to penetrate;
(j) determining a follow-up visit result group of the patient after observing the physical conditions of the patient; and
(k) prescribing a follow-up adjustment dosage for the patient based on the follow-up result group.

43. The method of claim 42, wherein the follow-up result group includes:
a follow-up result group 1, wherein the patient's penis is less than 50% rigid and the patient is unable to penetrate;
a follow-up result group 2, wherein the patient's penis is 50-70%, partially rigid and rubbery and the patient is just able to penetrate;
a follow-up result group 3, wherein the patient's penis is 70-80% rigid and the patient can penetrate reasonably easy;
a follow-up result group 4, wherein the patient's penis is 80-100% rigid for less than 60 minutes and the patient can penetrate;
a follow-up result group 5, wherein the patient has prolonged erection or 100% erection for 3-6 hours, the erection is reduced spontaneously or by conservative measures, and the patient can penetrate; and
a follow-up result group 6, wherein the patient has priapism and is not responsive to conservative treatment and the patient can penetrate.

44. The method of claim 43, further comprising increasing the same visit dose by 75% as the follow-up adjustment dosage for premature ejaculation, for a patient in the follow-up result group 1.

45. The method of claim 43, further comprising increasing the same visit dose by 50% as the follow-up adjustment dosage for premature ejaculation, for a patient in the follow-up result group 2.

46. The method of claim 43, further comprising increasing the same visit dose by 25% as the follow-up adjustment dosage for premature ejaculation, for a patient in the follow-up result group 3.

47. The method of claim 43, further comprising prescribing the same visit dose as the follow-up adjustment dosage for premature ejaculation, for a patient in the follow-up result group 4.

48. The method of claim 43, further comprising changing from a prescription of a solution of PGE1, papaverine, phentolamine, and atropine in the ratio of 15:45:25:15, respectively, to only PGE1 and using 75% of the original volume of the same visit dose as the follow-up adjustment dosage for premature ejaculation, for patient in the follow-up result group 5.

49. The method of claim 43, further comprising prescribing 14 units of PGE1 as the follow-up adjustment dosage for premature ejaculation, for a patient in the follow-up result group 6.

50. A method for a same visit treatment of achieving and maintaining male erectile response regardless of state of mind, occurrence of ejaculation, or lack of sexual arousal of a patient, the method comprising:
(a) performing a diagnostic on the patient using physical conditions of the patient, selected from the group consisting of blood flow rate, rigidity of erection, a sustainability of an erection, occurrence of morning erection, duration of erectile dysfunction, or an ability to penetrate;
(b) determining an initial diagnostic group of the patient, selected from the group consisting of a premature ejaculation group, a mild erectile dysfunction, a moderate erectile dysfunction group, and a severe erectile dysfunction, wherein each diagnostic group has corresponding conditions to the physical conditions of the patient;
(c) identifying an age group of the patient;
(d) selecting a first test dose of a pharmaceutical composition, which corresponds to the age group and the diagnostic group of the patient, wherein the pharmaceutical composition is a solution with PGE1, papaverine, phentolamine, and atropine;
(e) administrating the first test dose of the pharmaceutical composition to the patient;
(f) observing the physical conditions of the patient after the administration of the first test dose, including the rigidness of the penis of the patient or whether the patient is able to penetrate;
(g) determining a result group of the patient after observing the physical conditions of the patient;
(h) prescribing a same visit dosage for the patient based on the result group;
(i) observing the physical conditions of the patient after the administration of the same visit dosage, including the rigidness of the penis of the patient and whether the patient is able to penetrate;
(j) determining a follow-up visit result group of the patient after observing the physical conditions of the patient; and
(k) prescribing a follow-up adjustment dosage for the patient based on the follow-up result group.

51. A method for a same visit treatment of achieving and maintaining male erectile response regardless of state of mind, occurrence of ejaculation, or lack of sexual arousal of a patient, the method comprising:
(a) performing a diagnostic on the patient using physical conditions of the patient, selected from the group consisting of blood flow rate, rigidity of erection, a sustainability of an erection, occurrence of morning erection, duration of erectile dysfunction, or an ability to penetrate;
(b) determining an initial diagnostic group of the patient, selected from the group consisting of a premature ejaculation group, a mild erectile dysfunction, a moderate erectile dysfunction group, and a severe erectile dysfunction, wherein each diagnostic group has corresponding conditions to the physical conditions of the patient;
(c) identifying an age group of the patient;
(d) selecting a first test dose of a pharmaceutical composition, which corresponds to the age group and the diagnostic group of the patient, wherein the pharmaceutical composition is a solution with PGE1, papaverine, phentolamine, and atropine;
(e) administrating the first test dose of the pharmaceutical composition to the patient;
(f) observing the physical conditions of the patient after the administration of the first test dose, including the rigidness of the penis of the patient or whether the patient is able to penetrate;
(g) determining a result group of the patient after observing the physical conditions of the patient;
(h) prescribing a same visit dosage for the patient based on the result group;
(i) observing the physical conditions of the patient after the administration of the same visit dosage, including the rigidness of the penis of the patient and whether the patient is able to penetrate;
(j) determining a follow-up visit result group of the patient after observing the physical conditions of the patient;
(k) prescribing a follow-up adjustment dosage for the patient based on the follow-up result group;
(l) observing the physical conditions of the patient after the administration of 100 units of a solution of PGE1, papaverine, phentolamine, and atropine in the amount of 6 μg/ml, 10.5 mg/ml, 2.0 mg/ml and 0.15 mg/ml, respectively, including the rigidness of the penis of the patient and whether the patient is able to penetrate;
(m) determining a resistant erectile dysfunction result group of the patient after observing the physical conditions of the patient; and
(n) prescribing a first adjustment dosage for resistant erectile dysfunction for the patient based on the resistant erectile dysfunction result group.

52. A method for a same visit treatment of achieving and maintaining male erectile response regardless of state of mind, occurrence of ejaculation, or lack of sexual arousal of a patient, the method comprising:
(a) performing a diagnostic on the patient using physical conditions of the patient, selected from the group consisting of blood flow rate, rigidity of erection, a sustainability of an erection, occurrence of morning erection, duration of erectile dysfunction, or an ability to penetrate;
(b) determining an initial diagnostic group of the patient, selected from the group consisting of a premature ejaculation group, a mild erectile dysfunction, a moderate erectile dysfunction group, and a severe erectile dysfunction, wherein each diagnostic group has corresponding conditions to the physical conditions of the patient;
(c) identifying an age group of the patient;
(d) selecting a first test dose of a pharmaceutical composition, which corresponds to the age group and the diagnostic group of the patient, wherein the pharmaceutical composition is a solution with PGE1, papaverine, phentolamine, and atropine;
(e) administrating the first test dose of the pharmaceutical composition to the patient;
(f) observing the physical conditions of the patient after the administration of the first test dose, including the rigidness of the penis of the patient or whether the patient is able to penetrate;
(g) determining a result group of the patient after observing the physical conditions of the patient;
(h) prescribing a same visit dosage for the patient based on the result group;
(i) observing the physical conditions of the patient after the administration of the same visit dosage, including the rigidness of the penis of the patient and whether the patient is able to penetrate;
(j) determining a follow-up visit result group of the patient after observing the physical conditions of the patient;
(k) prescribing a follow-up adjustment dosage for the patient based on the follow-up result group;
(l) observing the physical conditions of the patient after the administration of 100 units of a solution of PGE1, papaverine, phentolamine, and atropine in the amount of 6 μg/ml, 10.5 mg/ml, 2.0 mg/ml and 0.15 mg/ml, respectively, including the rigidness of the penis of the patient and whether the patient is able to penetrate;
(m) determining a resistant erectile dysfunction result group of the patient after observing the physical conditions of the patient; and
(n) prescribing a first adjustment dosage for resistant erectile dysfunction for the patient based on the resistant erectile dysfunction result group;
(o) observing the physical conditions of the patient after the administration of 100 units of a solution of PGE1, papaverine, phentolamine, and atropine in the amount of 10 μg/ml, 6.0 mg/ml, 2.5 mg/ml and 0.05 mg/ml, respectively, including the rigidness of the penis of the patient and whether the patient is able to penetrate;
(p) determining a second resistant erectile dysfunction result group of the patient after observing the physical conditions of the patient; and
(q) prescribing a second adjustment dosage for resistant erectile dysfunction for the patient based on the second resistant erectile dysfunction result group.
